(19) United States
(12) Patent Application Publication
(10) Pub. No.: US 2025/0312048 A1
(43) Pub. Date: Oct. 9, 2025

(54) PATIENT-SPECIFIC HUMERAL HEAD GUIDE DEVICE AND SYSTEMS AND METHODS FOR MANUFACTURING SAME

(71) Applicant: 3D LIFEPRINTS UK LTD., Liverpool (GB)

(72) Inventors: Henry Pinchbeck, Dubai (UA); David Jimenez-Cruz, Manchester (GB)

(73) Assignee: 3D LIFEPRINTS UK LTD., Liverpool (GB)

(21) Appl. No.: 18/625,864

(22) Filed: Apr. 3, 2024

(51) Int. Cl. A61B 17/15; A61B 17/00; A61B 34/10; A61F 2/30

(52) U.S. Cl. CPC A61B 17/151; A61F 2/30942; A61B 2017/00526; A61B 2034/108

(57) ABSTRACT

A patient-specific humeral head guide device may be designed and manufactured to contour the shape of the humeral head of the patient, as determined from the medical images. For example, a system may generate computer-assisted manufacturing (CAM) instructions based on one or more medical images of the shoulder of the patient. The CAM instructions may include G-code that causes a three-dimensional (3D) printer to manufacture a patient-specific humeral head guide device. The system may generate G-code for other various portions of the patient-specific humeral head guide device. For example, the system may generate instructions for manufacturing a patient-specific humeral head guide device to include a footprint (including a body and an extended footprint) that contours the humeral head, a plurality of fixation wire channels (for drilling to receive a respective fixation wire), a reaming channel, a resection channel (for cutting), and/or other portions described herein.

### FIELD OF THE INVENTION

The present disclosure relates to user-specific medical devices such as surgical guide devices and methods and systems of manufacturing them.

### BACKGROUND

Manufacturing devices is typically a standardized process in which a device is designed and based on a single design and appropriate manufacturing steps are defined. A standard device is generally mass manufactured which requires the design and manufacturing process to be fixed with little room for either design or manufacturing set-up changes. However, for certain user-specific devices (e.g., patient-specific medical devices) there is a requirement that each device is designed and manufactured individually based upon the user characteristics and requirements. As such, it may be difficult to manufacture patient-specific devices using a mass manufacturing process. The advent of commercial scale computer-aided manufacturing (CAM) such as three-dimensional (3D) printing has opened up the potential for mass manufacturing of user-specific devices but this opportunity requires controls to be put in place for both the design and manufacturing processes to ensure the devices consistently meet the user characteristics and requirements as well as relevant quality and regulatory specifications.

### SUMMARY

Patient-specific medical devices and systems and methods for manufacturing these devices are described. For example, a system may access and use patient-specific data to customize patient-specific medical devices. The patient-specific data may include medical images that are used to identify and contour anatomical features that are specific to the patient. The medical images may include a Computed Tomography (CT) scan image, a Magnetic Resonance Imaging (MRI) scan image, an X-ray image, sonogram and/or other image of the patient for medical purposes. In some embodiments, a scanning technology that can generate a three-dimensional topography of an anatomical feature such as a CT scan may enable more precise contouring of the medical devices around the anatomical feature in two or more planes.

In some embodiments, a patient-specific medical device may include a humeral head guide device. A humeral head guide device is a device that guides an osteotomy in which a portion or all of the humeral head is removed so that it can be replaced with a medical implant device. In these embodiments, the system may use patient data, such as medical images of the shoulder of the patient, to design and manufacture a patient-specific humeral head guide device. In this way, the patient-specific humeral head guide device may be designed and manufactured specifically based on a patient's anatomy. In some embodiments, the patient-specific humeral head guide device may be manufactured as a single-use device, washed and ready to be sterilized for use.

The system may use a hybrid design approach using a reliable guide footprint that may be designed and manufactured on a patient-specific basis, making the footprint of the humeral head guide device bespoke. This design improves the usability of the guide and reduces the material cost. Each humeral head guide device may be designed as a custom solution with a pre-defined protocol disclosed herein. In these embodiments, the humeral head guide device may not be based on a design template that is modified for each patient. Due to the flexibility in its design, the humeral head guide device and other patient-specific medical devices may be customized to suit requirements for version, inclination and implant type.

In some embodiments, the system may design a patient-specific humeral head guide device to contour the shape of the humeral head of the patient, as determined from the medical images. In this example, the system may generate CAM instructions based on one or more medical images that includes a medical image of a shoulder of the patient. The CAM instructions may include G-code that causes a three-dimensional (3D) printer to manufacture a patient-specific humeral head guide device. The system may generate G-code for other various portions of the patient-specific humeral head guide device. For example, the system may generate instructions for manufacturing a patient-specific humeral head guide device to include a footprint (including a body and an extended footprint) that contours the humeral head, a plurality of fixation wire channels (for drilling to receive a respective fixation wire), a reaming channel, a resection channel (for cutting), and/or other portions described herein.

The humeral head guide device may be designed to avoid soft tissue attachments, while using approved guide anchor sites. In some examples, the system may design and manufacture the footprint of the humeral head guide device to be contoured in at least one plane around the humeral head. In some embodiments, the system may design and manufacture the footprint of the humeral head guide device to be contoured in all three planes around the humeral head without being exposed to undercuts that make it difficult to attach. This allows quick and confident placement of the humeral head guide device.

In some embodiments, the humeral head guide device may be improved to provide accurate cutting for a humeral head osteotomy at a pre-planned cutting plane at the desired version and inclination, enable accurate fixation of the humeral head guide device in the correct orientation, and/or other advantages. For example, the humeral head guide device may be designed and manufactured to include a resection channel for humeral head osteotomy at a pre-planned cutting plane, at the desired version and inclination. The resection channel is made suitable for the surgeons preferred thickness oscillating saw blade.

In some embodiments, the humeral head guide device may be designed and manufactured to include a plurality of fixation wire channels (sometimes referred to as drilling channels) that are each configured to receive a fixation wire (such as a K-wire) to attach the humeral head guide device. In some embodiments, the humeral head guide device may be designed and manufactured to include an alignment channel that is configured to receive an intermedullary reaming rod. The alignment channel is aligned to the center of the humeral canal, allowing the operator (such as a human or robotic surgeon) to create a space in the canal along a pre-determined axis. This helps to avoid the implant being fixed at an incorrect angle. To ensure the humeral head guide device is fixed in the correct orientation, the anterior axis (perpendicular to the intercondylar elbow axis) is translated to one of the fixation wire channels. The operator can then visually confirm that the forearm is aligned correctly with the anterior axis.

The patient-specific humeral head guide device and its design and manufacturing may be improved to mitigate against various problems, such as placement for the humeral head guide device being inaccessible, the humeral head guide device is improperly placed but is believed to be properly placed, failure of the humeral head guide device, and/or other issues.

Placement of the humeral head guide device may be inaccessible because of the severity of the humeral or glenoid deformity, patient size (soft tissue mass), unexpected residual cartilage thickness, obstructions from soft tissue attachments-rotator cuff, and/or other causes. To mitigate these problems, the humeral head guide device may be designed and manufactured to take into account the soft tissue attachments, and provides tolerance between the bone and guide at these sites. The humeral head guide device is designed and manufactured to maintain a low profile as possible without sacrificing strength. This helps with anchoring the humeral head guide device and having visibility of how it is anchored to the bone. Additional tolerance is also given at the articulating surface to allow for unexpected cartilage that cannot be accounted for during the segmentation of the CT scan.

The humeral head guide device may be improperly placed, but believed to be properly placed, which may result in an unplanned resection plane, risk of damage to the rotator cuff, over-stuffing the resection, incongruity between the humeral and glenoid components, leading to implant ware/failure or dislocation, and/or other issues. To mitigate these problems, the humeral head guide device may be designed and manufactured based on a detailed 3D digital model, with different view options to illustrate the resection plane. Additionally, or alternatively, the design report may include an indication of appropriate guide position and landmarks to be used for the correct guide orientation. The humeral head guide device may also be manufactured to include a marking or other indication (such as “ANT”) on a fixation wire channel configured to receive a fixation wire that aligns with the anterior axis of the humerus. Thus, this marked fixation wire channel can be checked with the patient's forearm to ensure the guide is seated correctly (as previously noted).

The humeral head guide device may fail such as by breaking at a portion such as the resection channel. This failure may lead to an ambiguous cutting plane direction, fragments of the humeral head guide device falling into the surgical site, damage surrounding tissue, injure surgical staff, and/or other issues. To mitigate these problems, the humeral head guide device may be designed and manufactured based on simulation testing using 3D printed models of suitable anatomy to ensure the materials and geometry used for the humeral head guide device are suitable for intraoperative use. For example, the materials may include a biocompatible photopolymer resin with a range for elongation break of approximately 12-33% and range of approximately 0.99-2.08 GPa for Young's modulus. These or other material characteristics for other materials, along with appropriate design, offers a suitable solution to avoid guide breakage. Alternatively, or additionally, the humeral head guide device may be designed and manufactured to improve robustness and resilience, such as forming the thickness of the resection channel wall thickness to be at least approximately the same thickness of the resection channel, setting a minimum thickness of the humeral head guide device such as approximately 3 millimeters (mm), configuring a tolerance of the resection channel to prevent the blade (cutting device inserted into the resection channel) from “sticking” and shaking the guide free/breaking, and/or other design protocol techniques.

Due to the limited intra-operative access the patient-specific humeral head guide device is designed as low profile as possible, with a window for visibility of the underlying anatomy. This design approach is to ensure that the patient-specific humeral head guide device is seated correctly on the humeral head. In some embodiments, due to the complex geometry of the anatomy in this region, the patient-specific humeral head guide device is designed and manufactured to fit in only one orientation. Once positioned, the operator (such as a surgeon or robotic assistive device) can then fix the guide using a number (such as three) fixation wires such as K-wires.

### BRIEF DESCRIPTION OF THE DRAWINGS

FIG. 1 is a block diagram illustrating data exchange and interactions between different entities via a system for controlling a user-specific device manufacturing process, in accordance with one or more embodiments.

FIG. 2 is a schematic illustration of a system configured for controlling a manufacturing process of user-specific devices, in accordance with one or more embodiments.

FIG. 3A is a schematic illustration of an anterior view of a humeral head showing an inclination 310 for fitment of an implant for total shoulder replacement, in accordance with one or more embodiments.

FIG. 3B is a schematic illustration of a view of a humeral head from a top-down perspective relative to the anatomical axis 302 shown in FIG. 3A showing a retroversion 320 for fitment of an implant for total shoulder replacement, in accordance with one or more embodiments.

FIG. 4 is a schematic illustration 400 of an incision 410 for total replacement surgery, in accordance with one or more embodiments.

FIG. 5 is an example of a patient-specific humeral head guide device 500 on a humeral head 501, in accordance with one or more embodiments.

FIG. 6 is a schematic illustration of anatomical landmarks for an interim anterior axis protocol, in accordance with one or more embodiments.

FIG. 7 is a schematic illustration of the definition of the humeral sagittal plane on a proximal humerus (Anterior Axis Orientation) to determine a superior resection plane used for a resection channel, in accordance with one or more embodiments.

FIG. 8 is a schematic illustration showing a distance between the superior resection plane 801 to lateral, in accordance with one or more embodiments.

FIG. 9A shows a schematic illustration of a seat and a plate of a two-part design of a humeral head guide device, in accordance with one or more embodiments.

FIG. 9B shows a schematic illustrations of different perspective views of an assembly of the seat and plate portions illustrated in FIG. 9A, in accordance with one or more embodiments.

FIG. 9C shows a schematic illustration of a humeral head guide device featuring a two-part design with open cutting surface attached to a humeral head, in accordance with one or more embodiments.

FIG. 10 shows schematic illustrations of different perspective views of another embodiment of a humeral head guide device, in accordance with one or more embodiments.

FIG. 11 shows schematic illustrations of different perspective views of another embodiment of a humeral head guide device, in accordance with one or more embodiments.

FIG. 12 shows schematic illustrations of different perspective views of another embodiment of a humeral head guide device, in accordance with one or more embodiments.

FIGS. 13A-13C show schematic illustrations of different perspective views of another embodiment of a patient-specific humeral head guide device, in accordance with one or more embodiments.

FIGS. 14A-F show schematic illustrations of different perspective views of another embodiment of a patient-specific humeral head guide device, in accordance with one or more embodiments.

FIG. 15 illustrates a flow diagram of an example method for identifying a custom resection plane for a patient-specific humeral head guide device, in accordance with one or more embodiments.

FIG. 16 illustrates a flow diagram of an example method for designing a patient-specific humeral head guide device, in accordance with one or more embodiments.

FIG. 17 illustrates a flow diagram of an example method for manufacturing a patient-specific humeral head guide device, in accordance with one or more embodiments.

FIG. 18 illustrates a flow diagram of an example method for designing a patient-specific device, in accordance with one or more embodiments.

### DETAILED DESCRIPTION

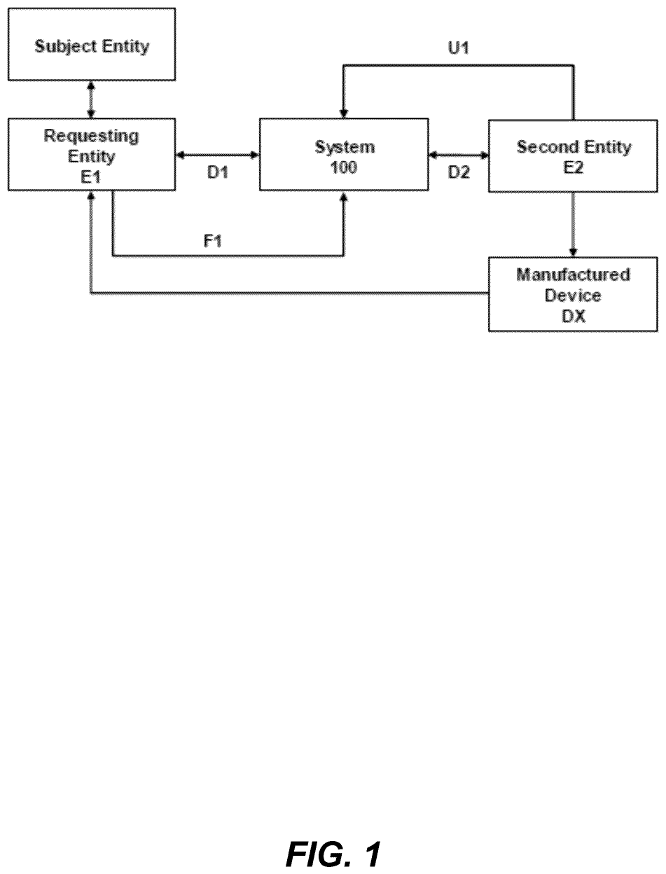

FIG. 1 is a block diagram illustrating data exchange and interactions between different entities via system 100 for controlling a user-specific device manufacturing process, in accordance with one or more embodiments.

In an embodiment, the system 100 may be configured to perform a regional configuration. For example, a requesting entity's country details may be stored in a customer database. So, when a new device request is received the system may preconfigure a workflow for any regulatory specification in the corresponding requesting entity's territory.

In an embodiment, the system 100 may perform a case type or model selection. In an embodiment, when the requesting entity selects a type of case from a new device request form, the system automatically determines a number and type of devices for completing the request. For example, for a new request for a craniomaxillofacial (CMF) implant, the system 100 may generate or suggest three devices—an anatomical model for diagnostics, a surgical cutting guide for intra-surgical purposes and the implant itself.

In an embodiment, the system 100 may be configured to determine manufacturing information for the new device request. In an embodiment, the system 100 matches a new device request with a 3D printer, a material, or both. The 3D printer may include a device that is suitable to manufacture a new device, such as various examples of the patient-specific humeral head guide device disclosed herein, depending on the type of material used for 3D printing. For example, the 3D printer may include, without limitation, a Stereolithography (SLA) printer, a Selective laser sintering (SLS) printer, a Fused deposition modeling (FDM) printer, a Direct metal laser sintering (DMLS) printer, and an Electron beam melting (EBM) printer.

In an embodiment, the system 100 comprises a rules database having a list of rules based on a type of procedure and nature of the device requested. The rules are used to determine a preferred manufacturing technology (e.g., 3D print technology and raw material) to achieve a suitable product for the procedure involved. In some cases, manufacturing is subcontracted for particular products and such subcontracting information is also provided and tracked as a part of the workflow, and the workflow updated accordingly.

In an embodiment, the system 100 may be configured to perform resource selection based on the requested device. For example, one or more engineers may be assigned to the requesting entity in the customer database. The engineer's workload and customer assignments may be tracked within the system 100. The system 100 may include an algorithm for resource allocation, where the algorithm may select and assign a most appropriately qualified engineer with suitable capacity to the newly request device, and the engineer may be notified about the assignment by email.

In an embodiment, the system 100 may be configured to determine a workflow selection based on information related to the requested device. For example, workflows for each device category with a series of deliverables to be tracked are stored within a project template database. As a device type is selected, the corresponding workflow is automatically assigned in the system 100. This ensures that engineers time is controlled appropriately on each project (e.g., for use with capacity management) and also that any corresponding regulatory specifications assigned based on the territory can be outlined and monitored in the same workflow.

In an embodiment, the system 100 selects one or more devices based on data received from the requesting entity. For example, a use case (e.g., an intended use) identified in a device request form (from a list of pre-determined device types) may be used to match a set of rules stored within a project database. A logic is then applied to a new project to assign a status (e.g., a patient specific medical device, educational device, etc.) to the device. This selection step is important because although the inputs and outputs of a project can be similar the use case can determine whether the requested device is used for diagnostic or treatment purposes. Based on the purpose, the requested device may be classified as a user-specific device as opposed to an educational or research purpose device which would exempt the requested device from the medical device classification.

In an embodiment, the system 100 facilitates dynamic generation and receiving of user-specific device information D1 from a requesting entity E1 to conform to requirements of a subject entity, determining manufacturing related information D2 based on the user-specific information D1, and augmenting the manufacturing decisions with the user-specific device information D1. The system 100 may transmit the device information D1 to a second entity E2 for designing and/or determining manufacturing steps. As an example, the second entity E2 may be a designing entity that determines the shape, size, material, etc. based on the information D1 received from the requesting entity E1. As another example, the second entity E2 may be a manufacturing entity that determines the manufacturing workflow based on the design of the user device received from the designing entity and information received from the requesting entity E1. The system 100 may determine manufacturing related information D2 from databases and/or the second entity E2 that may be translated into questions for the requesting entity E1 to dynamically collect use-specific information.

For example, the device information D1 may include intended use and desired outcome based on which potential list of devices may be determined that achieve the desired outcome. The system 100 further receives feedback information F1 from the requesting entity E1 and based on the feedback information F1, the second entity E2 updates the design/manufacturing related information D2 to generate the updated information U1. Once, the updated information U1 is approved by the requesting entity E1, the second entity may manufacture the user-specific device DX and ship it to the requesting entity E1.

In an embodiment, the system 100 for controlling design and manufacturing of the user-specific device may be configured to prompt the requesting entity E1 to provide appropriate user-specific information (e.g., a desired outcome, a type of application, etc.), and suggest a list of devices and their characteristics based on the user-specific information that can potentially solve the user issue. The system 100 implements an algorithm configured to determine the list of devices, and/or manufacturing flow or steps based on information (e.g., intended use or desired outcome) in the user-specific information. The algorithm comprises several decision points that lead to selecting one device design over another, selecting one manufacturing flow over another, or other functions based on the user-specific information. In an embodiment, one or more predictive models may be executed to predict estimated delivery times, cost, resources, etc. The predictive model also enables updating of the delivery times, resource allocation, and costs for any changes that may occur based on the feedback from the requesting entity. In an embodiment, the model may be a linear model, quadratic model, or other mathematical models.

In an embodiment, the requesting entity E1 may be any person with knowledge about the structure and functions of the body parts (e.g., anatomy of human body parts), procedures required to implant a device in the body part, pre-planning required to implant the device, devices that may be employed to fix the issues related to the body part, or having other information related to the device and a user. For example, the requesting entity may be a healthcare professional such as doctors, surgeons, clinicians, or others who can provide information about anatomy of a subject entity receiving a treatment or a medical device. In an embodiment, the second entity E2 is different from the requesting entity E1. The second entity E2 may be any person involved in designing and/or manufacturing of the device based on the inputs from the requesting entity E1. For example, the second entity E2 may be engineers, designers, regulatory compliance officers, production manager, or other personnel related to design, manufacturing and delivery of devices.

In some embodiments, based on the user-specific inputs (e.g., structural and procedural constraints), system 100 generates an interactive digital model associated with one or more user-specific devices prior to manufacturing for review by the requesting entity. Since, the user-specific device may depend on a structure of the body part, the review of the interactive digital model of the device provides a visual guidance to the requesting entity on how the device will interact or correspond to the structure of the body part (e.g., anatomy of human body part). Based on the interactive digital model, the requesting entity may determine whether the device will provide desired results to the user upon using the device in reality. Such determination may not be done by the second entity since the second entity may not have knowledge about how to analyse impact of changes to the design, and whether desired results may be obtained due to such changes. Upon review, the requesting entity may manipulate or provide feedback on the interactive digital model to accurately point out the changes, if any, as needed. In this way, the interactive digital model advantageously captures additional user-specific data (such as structural and procedural constraints) and incorporates it directly on the device design.

When such feedback is transmitted to the second entity, changes to the device design can be implemented according to user-specific requirements. Advantageously, providing and accessing the feedback changes directly from the interactive digital model provides enables the system to transmit user-specific data to the second entity, as well as make changes to design/manufacturing workflow (e.g., a sequence of tasks to be performed by one or more entities at a given point in time) corresponding to the feedback changes. Such visual guidance is beneficial to understand and incorporate the requested changes in the device design, the manufacturing process, or both. Thus, the system enables generation of the manufactured device (e.g., DX in FIG. 1) having a structure and functions that are user-specific. Accordingly, the manufactured device will be more accurate and satisfy user-specific needs (structurally and functionally) compared to a mass manufactured device. Also, waste or remanufacturing of a user-specific device can be prevented.

Another advantage of the system 100 is that it improves inter process communication more efficient by accessing and sharing information based on user-specific data from different databases such as databases configured with user-specific information, device information, manufacturing information, or other information stored in different databases. The system 100 facilitates generating an improved solution (e.g., in terms of devices, procedures and manufacturing process) by making the interaction between the requesting entity and the second entity more efficient resulting in a more efficient overall manufacturing process. For example, timely feedback and changes ensure timely manufacture and delivery of medical devices to the requesting entity so that a subject entity such as a patient may receive a desired treatment in a reasonable time. In an embodiment, efficient interactions refer to less number of interactions facilitated via dynamic input generation to capture most relevant user-specific information, rather than generic information that may require several follow-up questions from the second entity. In an embodiment, efficient interaction refers to having all the information about the overall manufacturing process accessible via a single client portal of the technology platform, rather than logging into different portal to securely access desired information about requested devices. For example, in existing systems, the requesting entity may need to correspond with the designing entity or the manufacturing entity over emails several times, open different files on different software or login-based portals, track the delivery from a different portal, etc. This leads to highly inefficient process wasting valuable time of the requesting entity (e.g., doctors, surgeons). Also, the user-information may not get communicated in an effective manner to the designing entity of the manufacturing entity.

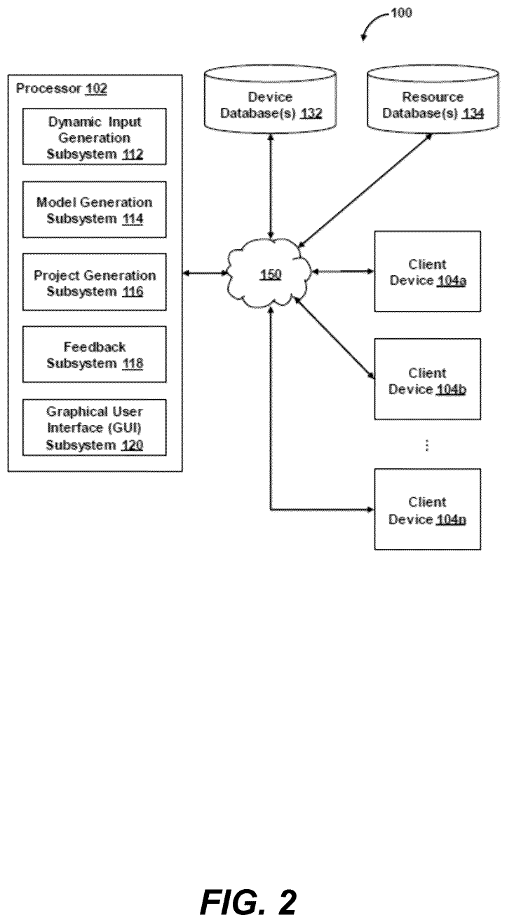

In an embodiment, as shown in FIG. 2, the system 100 may include processor 102, client device 104 (or client devices 104a-104n), device database 132, resource database 134, or other components. In an embodiment, the client device 104a may be accessed by one or more requesting entities (e.g., surgeons, clinicians, etc.) that are requesting one or more user-specific devices for a user. The one or more requesting entities are knowledge about analyzing user problems and solutions of a subject entity. For example, the requesting entity can understand and analyze implementation and functioning of user-specific devices in a given environment (e.g., anatomy of the subject entity), or operating procedures using the user-specific devices. In an embodiment, the requesting entity may be located in a first location and not knowledgeable about the design and manufacturing processes of the one or more user-specific devices. In an embodiment, another client device 104b may be accessed by one or more second entities (e.g., CAD designers, engineers, manufacturing experts, tool suppliers, etc.) that are in charge of designing, manufacturing, or both of the one or more user-specific devices. The second entity may be located at a second location and not knowledgeable about analyzing user problems (e.g., treatment, fracture, disease, etc.). As such, the second entity may not be able to analyze how any changes to the user-specific device may improve the solution or negatively impact the user problems.

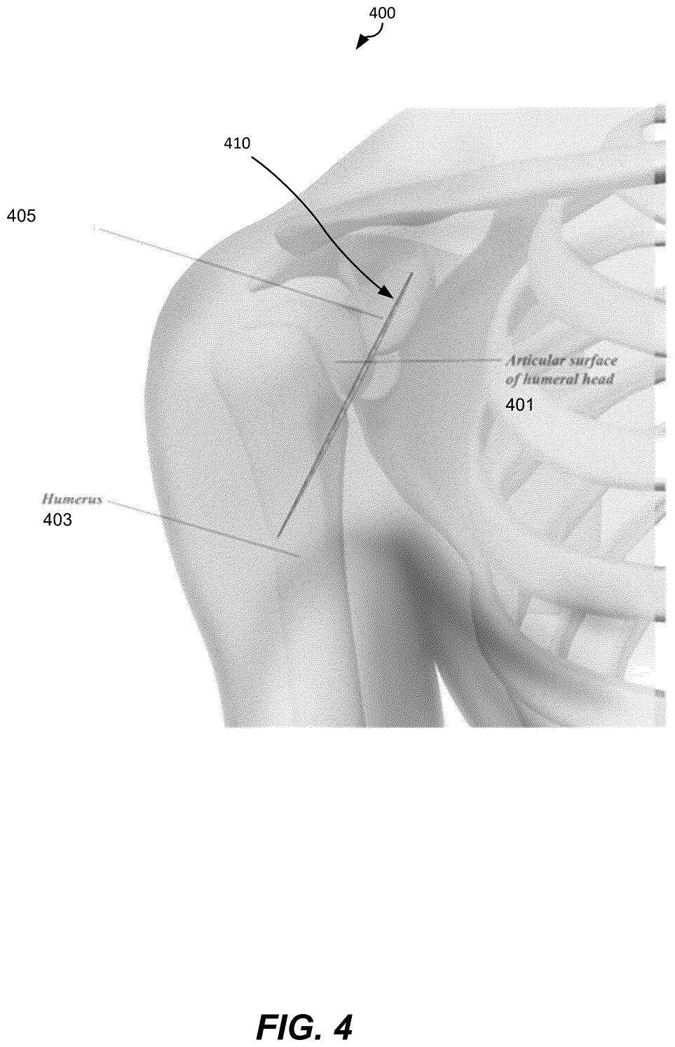

In an embodiment, the system 100 is configured to integrate different types of software applications and algorithms (e.g., see FIG. 4) to generate a manufacturing workflow specific to the user-specific device. The manufacturing workflow may include a sequence of tasks to be performed by one or more entities at a given point in time. The tasks may be determined based on the user-specific information associated with a requested user-specific device. Thus, the system 100 enables automatic determination of specific workflows suitable for a requested user-specific device. For example, generating the workflow includes generating appropriate questions or information via the dynamic input generation subsystem 112, generating a device design based on the received user-specific information via the model generation subsystem 114, receiving a feedback and approval via the feedback subsystem 118, and/or generating manufacturing steps for manufacturing the user-specific devices via a project generation subsystem 116.

Processor 102 may include dynamic input generation subsystem 112, model generation subsystem 114, project generation subsystem 116, feedback subsystem 118, graphical user interface subsystem 120, or other components. Each client device 104 may include any type of mobile terminal, fixed terminal, or other device. By way of example, client device 104 may include a desktop computer, a notebook computer, a tablet computer, a smartphone, a wearable device, or other client device. Users may, for instance, utilize one or more client devices 104 to interact with one another, one or more servers, or other components of system 100. It should be noted that, while one or more operations are described herein as being performed by particular components of processor 102, those operations may, in some embodiments, be performed by other components of processor 102 or other components of system 100. As an example, while one or more operations are described herein as being performed by components of processor 102, those operations may, in some embodiments, be performed by components of client device 104.

The client devices 104a-104n may be configured to display a graphical user interface that displays, receives, and/or allows manipulation of user-specific information. The graphical user interface manages the interaction between a computer system and different entities through graphical elements such as windows on a display. The graphical user interface enables efficient exchange of different type of information between different entities. Based on the information exchanged via the graphical user interface, the system 100 determines a sequence of tasks to be performed by different entities or the computer system in order to control the designing and manufacturing of user-specific devices.

In an embodiment, the information exchanged via the graphical user interface may be related to a structure and functions of the user-specific devices, designing task of the user-specific devices, feedback task related to the designed devices, and/or manufacturing tasks of the user-specific device. These tasks may be performed by different entities at different point in time. As such, an integration of various tasks into a manufacturing workflow is beneficial so that each entity can view, analyze, and provide timely feedback before manufacturing the actual user-specific device. In an embodiment, the processor 102 includes exemplary subsystems that enables seamless integration of these process so that end-to-end manufacturing process of user-specific devices may be controlled by the requesting entity and/or the second entity.

In some embodiments, the dynamic input generation subsystem 112 may be configured to generate a data input request to receive user-specific inputs from the requesting entity E1. In an embodiment, the generated data input request conforms with requirements of a subject entity. In an embodiment, the data input request may include user-specific questions that may be displayed via the graphical user interface subsystem 120 to the requesting entity E1 and/or other users having secured access to the system 100. In an embodiment, the dynamic input generation subsystem 112 may request a high-level information (e.g., an intended use, a condition of the user, a procedure in which the device may be used, a desired outcome, etc.) from the requesting entity E1 to determine a type or category of devices desired by the requesting entity E1 that may potentially improve issues faced by a user (e.g., the requesting entity or a subject entity). Once the type or category of devices is determined, the dynamic input generation subsystem 112 may generate additional questions regarding user-specific data to modify or design one or more devices for the requesting entity E1. As an example, the dynamic input generation subsystem 112 may securely communicate with a device database 132 to extract device information related to one or more devices that may help with the user-specific problems and request additional information from the requesting entity E1 based on the extracted device information.

In some embodiments, the system 100 may receive the user-specific inputs via a graphical user interface that is dynamically configured to include input fields to receive user-specific data (e.g., structural or procedural constraints). In an embodiment, the system 100 may dynamically update one or more input fields based on the user-specific data entered by the requesting entity so that appropriate information related to one or more user-specific devices may be collected and used during the designing and/or manufacturing process. In some embodiments, upon receiving the user-specific inputs, the system 100 may transmit the user-specific inputs to a second entity for designing of the one or more devices.

In some embodiment, system 100 may be configured to manufacture a patient-specific medical device. A medical device may be any device intended to be used for medical purposes. Medical devices benefit patients by helping health care providers (e.g., a surgeon) diagnose and treat patients, helping patients overcome a medical condition such as sickness or disease, and improving the patient's quality of life. Medical devices are associated with several constraints when using a device for medical purposes including structural, functional, regulatory compliance associated with a geographical location e.g., country, state, or other constraints related to treatment or surgery procedures. As an example, the medical device may be an anatomical model of the body part of a subject entity (e.g., a patient), a surgical guide to be used for a surgery by a requesting entity (e.g., a surgeon); or an implant for the body part of the subject entity. As additional example, one or more user-specific device may be a devices used to improve a medical condition of a patient such as a patient specific face mask. Patients may have unique characteristics such as body part structures, medical issues, etc. which constraints the design and manufacturing of the patient-specific device.

In an embodiment, the device characteristics may be at least one of: a particular material based on the intended use of the device; a customized geometry to fit the structure of the body part during a particular treatment cycle; and a sub-component or a particular area of interest of the structure of the body part. In an embodiment, the particular material has one or more material property comprising: biocompatibility, flexibility, durability, transparency, utility, life-like appearance, or a combination of properties.

In an embodiment, the intended use of the device comprises at least one of: a pre-surgical planning by a requesting entity; an intra-surgical device used by the requesting entity; visual communications (e.g., with a group or a patient); surgical simulation prior to the intended procedure; a post-surgical procedure to be performed by the requesting entity or the subject entity; and an implantation in the body part of the subject entity.

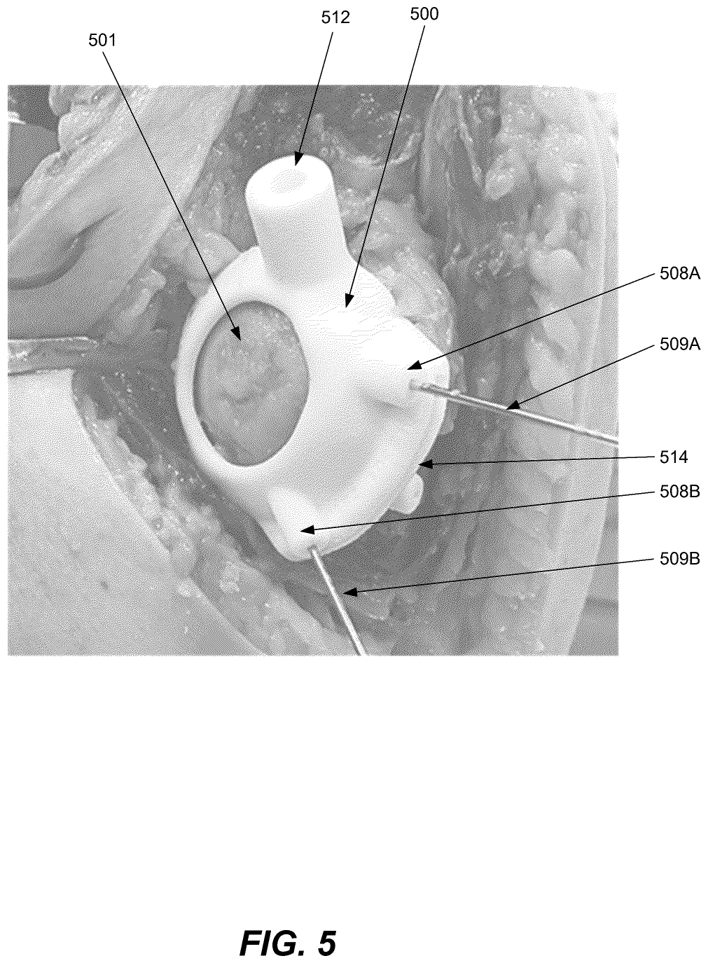

In an embodiment, the device generated by the systems illustrated in FIGS. 1 and 2 is a patient-specific humeral head guide device such as devices illustrated in FIGS. 5-14 (including any sub-parts A, B, and so forth). For example, one or more of the systems illustrated in FIGS. 1 and 2 (a “disclosed system”) may design a patient-specific humeral head guide device. In particular, a disclosed system may access one or more medical images of a patient. The patient-specific humeral head guide device may be used by an operator to perform a partial or complete humeral head osteotomy, such as for total shoulder replacement surgery. The operator may be a human surgeon, a surgical machine such as a surgical robotic device, or other operator that may perform the surgery.

A disclosed system may generate computer-aided manufacturing instructions, such as 3D printing G-code to print the patient-specific humeral head guide device. In this example, the computer-aided manufacturing instructions may include instructions to print the patient-specific humeral head guide device (and various portions thereof described herein).

The 3D printer may use various materials that are suitable for medical device guides. These materials may be approved by a regulatory agency. For example, the material may include a biocompatible photopolymer resin with a range for elongation break of approximately 12-33% and range of approximately 0.99-2.08 GPa for Young's modulus. In an embodiment, to facilitate single-use designs, a material such as Nylon 12 (also referred to as polyamide 12) may be used. Nylon 12 is generally considered biocompatible and is lightweight and flexible. However, other 3D materials that are suitable or approved for used as medical devices may be used. These other materials may include, without limitation, Titanium and Titanium Alloys, Stainless Steel, Cobalt-Chromium Alloys, and Polyether Ether Ketones.

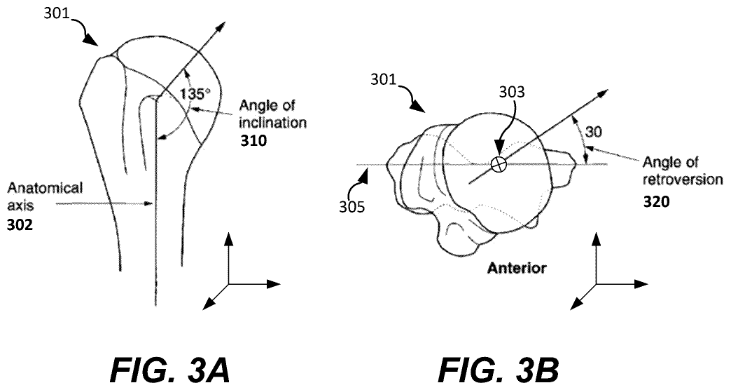

FIG. 3A is a schematic illustration of an anterior view of a humeral head showing an inclination 310 for fitment of an implant for total shoulder replacement, in accordance with one or more embodiments. The anterior view is a view from the perspective of viewing a patient's anterior (front). The angle of inclination 310 is approximately 135 degrees relative to an anatomical axis 302. In some embodiments, the angle of inclination 310 is between approximately 130 degrees and approximately 130 and approximately 155 degrees relative to an anatomical axis 302.

FIG. 3B is a schematic illustration of a view of a humeral head from a top-down perspective relative to the anatomical axis 302 shown in FIG. 3A showing a retroversion 320 for fitment of an implant for total shoulder replacement, in accordance with one or more embodiments. The retroversion 320 is approximately 30 degrees measured from the anatomical axis 302 relative to a horizontal plane 305. In some embodiments, the retroversion 320 is between approximately 0 and approximately 30 degrees. In some embodiments, the retroversion 320 is between approximately 20 and approximately 30 degrees.

Referring to both FIGS. 3A and 3B, total shoulder replacement (TSR) requires the removal and replacement of some or all of the humeral head 301 with either a spherical (anatomic) or flat plate (reverse) implant. Anatomic implants are designed to replicate the natural anatomy of the shoulder joint. They are typically used in patients with a healthy rotator cuff. Reverse implants are designed to provide stability and function in patients with a damaged rotator cuff. The ball and socket of the joint are reversed, so that the deltoid muscle group provides the power for movement instead of the rotator cuff.

The orientation of these implants is important to joint stability and implant fixation. The shoulder joint has a natural retroversion of between approximately 0-40 degrees. When replacing the shoulder, the stability of the joint can be improved by increasing the retroversion at the humeral head 301. The inclination of the implant is primarily decided by the implant manufacture at the time of the implant design. This is particularly true for stemmed type implants which enter into the medullary canal of the humerus.

FIG. 4 is a schematic illustration 400 of an incision 410 for TSR, in accordance with one or more embodiments. As illustrated, in some embodiments, the incision 410 is made across at least a portion of the articular surface of the humeral head 401, the humerus 403, and the coracoid process 405. To prepare the humerus 403 for joint replacement the head is resected at angles shown in FIGS. 3A and 3B. Performing this osteotomy accurately, intraoperatively has a heavy reliance on the surgeon's experience and the condition of the anatomy. To improve outcomes, a patient-specific humeral head guide is designed and manufactured (such as by using custom three-dimensional (3D) printing techniques) to provide a preplanned cutting plane for the osteotomy. This gives the surgeon the ability to approve a resection prior to surgery, reducing the intra-operative planning time and risk of error. Embodiments of humeral head guide designs are illustrated in FIGS. 5 and 9-14 (including any sub-parts of these Figures).

FIG. 5 is an example of a patient-specific humeral head guide device 500 on a humeral head 501, in accordance with one or more embodiments. The patient-specific humeral head guide device 500 includes a plurality of fixation wire channels 508 (illustrated as fixation wire channels 508A and 508B), a resection channel 514, and a reaming channel 512. Each of the plurality of fixation wire channels 508 are configured to receive a respective fixation wire 509 (illustrated as fixation wires 509A and 509B). The resection channel 514 is configured to receive a cutting blade used to remove all or portion of the humeral head 501 in preparation for an implant to be implanted in the patient.

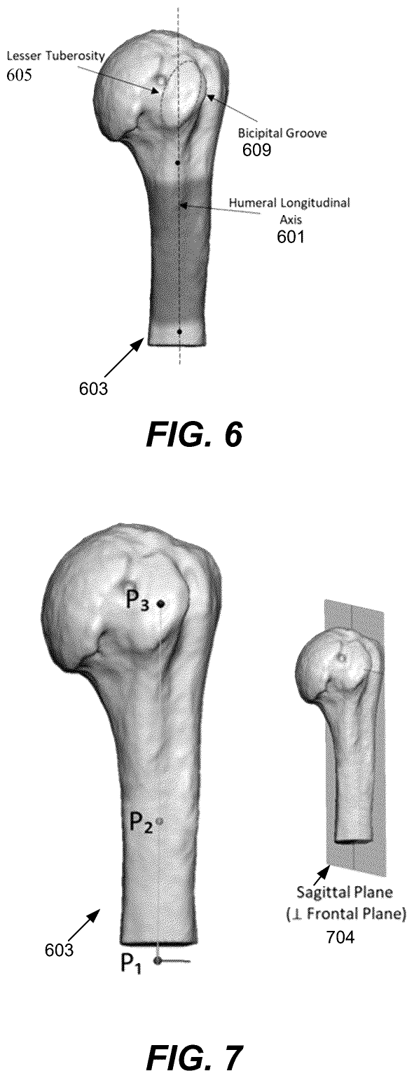

To generate the patient-specific humeral head guide device 500, a cadaveric shoulder specimen was scanned using the Insight Surgery Scanning Protocol. This was repeated for another cadaveric shoulder specimen for another patient-specific humeral head guide device 500 (only one is illustrated in FIG. 5). A patient-specific humeral head guide device 500 was designed for each cadaver using the interim anterior axis protocol, an embodiment of which is illustrated in FIG. 6. Cadaveric shoulder specimens were used for testing the guide designs to illustrate the following aspects of the design: surgical access, soft-tissue attachments, anatomical landmarks for guide orientation, and suitable and non-suitable areas for footprint coverage.

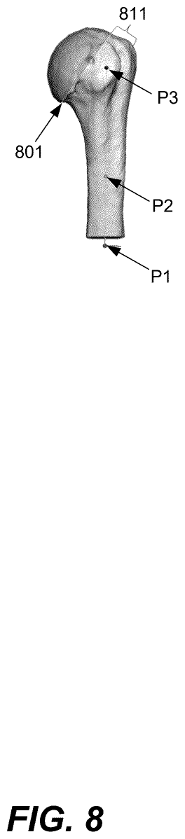

FIGS. 6-8 will be described together, with like reference numbers indicating the same elements throughout. FIG. 6 is a schematic illustration of anatomical landmarks for an interim anterior axis protocol, in accordance with one or more embodiments. The interim anterior axis protocol is defined based on the humeral longitudinal axis 601 and lesser tuberosity 605 to estimate the sagittal plane of the humerus 603 and the orientation of the anterior axis. FIG. 7 is a schematic illustration of the definition of the humeral sagittal plane 704 on a proximal humerus (Anterior Axis Orientation) 703 to determine a superior resection plane 801 used for a resection channel, in accordance with one or more embodiments. FIG. 8 is a schematic illustration showing a distance 811 between the superior resection plane 801 to lateral, in accordance with one or more embodiments.

Referring to FIG. 6, the humeral longitudinal axis 601 is determined based on a virtual cylinder around the humeral shaft. The cylinder may be a “best fit” cylinder around the humeral shaft. A 3D plane is determined by selecting the two points (illustrated in FIG. 7 as points P1 and P2) that define the humeral longitudinal axis 601 and a colinear point (illustrated in FIG. 7 as point P3) at the center of the lesser tuberosity 605. The resultant 3D plane is a humeral sagittal plane 704 and the anterior axis is coplanar to it. Alternatively or additionally, in some embodiments, the anterior axis may be defined by the bicipital groove axis in the transverse plane that forms an average of approximately 48 degree angle with the humeral Transepicondylar Axis (TEA), which is also perpendicular to the anterior axis. This 48 degree angle is an average, and may vary based on patient-specific physiology.

Referring to FIG. 7, from the humeral sagittal plane 704, a new plane can be positioned at approximately 135 degrees of inclination from the humeral longitudinal axis 601 and approximately 0-40 degrees of retroversion (in some examples, approximately 30 degrees) from the anterior axis. The new plane should exit the humerus, posteriorly, superior (1-2 mm) to the teres minor and infraspinatus attachment sites. This new plane will be the resection plane (illustrated in FIG. 8 as the superior resection plane 801) used for the guide design. FIG. 8 is a schematic illustration showing a distance 811 between the superior resection plane 801 to lateral, in accordance with one or more embodiments. The distance 811 may vary depending on the specific physiology of the patient. Thus, the distance 811 may be customized for a given patient. In some embodiments, in general, the distance 811 may be between approximately 9-14 millimeters (mm). In some embodiments, the distance 811 is approximately 10 (mm).

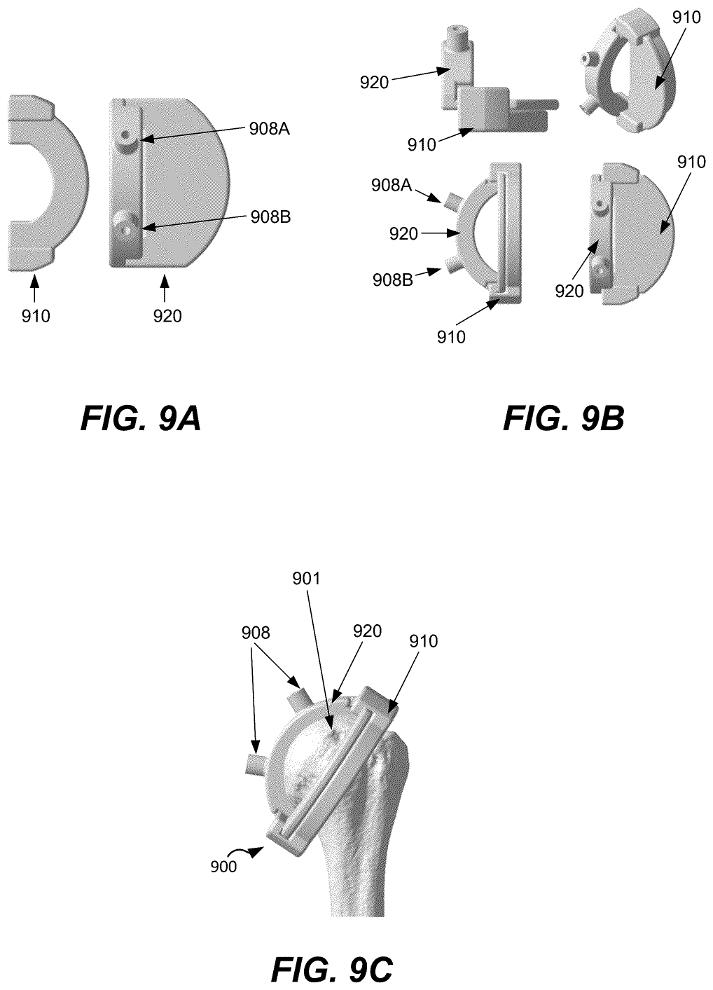

FIG. 9A shows a schematic illustration of a seat 910 and a plate 920 of a two-part design of a humeral head guide device, in accordance with one or more embodiments. The seat 910 sits against the bone at the head-neck junction. The plate 920 forms an open face cutting surface includes a plurality of guide wire channels 908, although other numbers of guide wire channels may be used).

FIG. 9B shows a schematic illustrations of different perspective views of an assembly of the seat 910 and plate 920 portions illustrated in FIG. 9A, in accordance with one or more embodiments. The plate 920 slots into the seat 910 giving the guide it's alignment to the planned osteotomy plane. The plate 920 is then fixed into place onto the humeral head using anti-parallel guide wires, such as Kirschner wires (also called k-wires), through respective guide wire channels 908.

FIG. 9C shows a schematic illustration of a humeral head guide device 900 featuring a two-part design with open cutting surface attached to a humeral head 901, in accordance with one or more embodiments. The humeral head guide device 900 (the assembly of the seat 910 and plate 920) fits on the anterior aspect of the humeral head neck junction. The humeral head guide device 900 fixes on the resection side only for ease of removal.

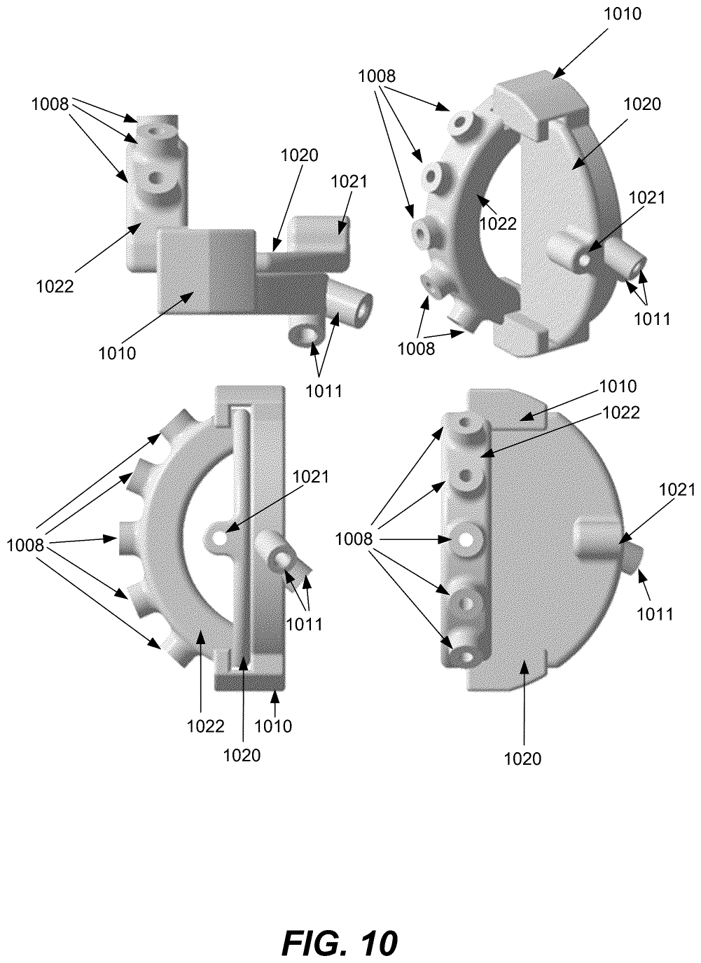

FIG. 10 shows schematic illustrations of different perspective views of another embodiment of a humeral head guide device 1000, in accordance with one or more embodiments. Review of embodiment shown in FIGS. 9A-9C highlighted the limitations of the options for fixation of a guide to the humerus. A need was also realized to verify the alignment with the anterior axis using the patient's forearm as a reference.

Relative to the embodiment shown in FIGS. 9A-9C, more guide wire channels 1008 for drilling around an armature 1022 of the plate 1020 is provided to give multiple options for fixation. This allows for limitations in the surgeons approach caused by soft tissue.

A fixation channel 1021 is included on the resection side of the guide cutting surface, perpendicular to the guide wire channels 1008. Alignment channels 1011 (such as at 20 and 30 degrees) were added to the seat 1010 to allow the surgeon, using a K-wire, to check the orientation of the humeral head guide device 1000 in relation to the patient's forearm. Such an alignment channel 1011 may be marked (such as with the letters “ANT” or other indication) on the humeral head guide device 1000 to indicate that a K-wire inserted therein can be used to verify alignment with the forearm. This label may be etched, printed, or otherwise visually noted on the humeral head guide device 1000.

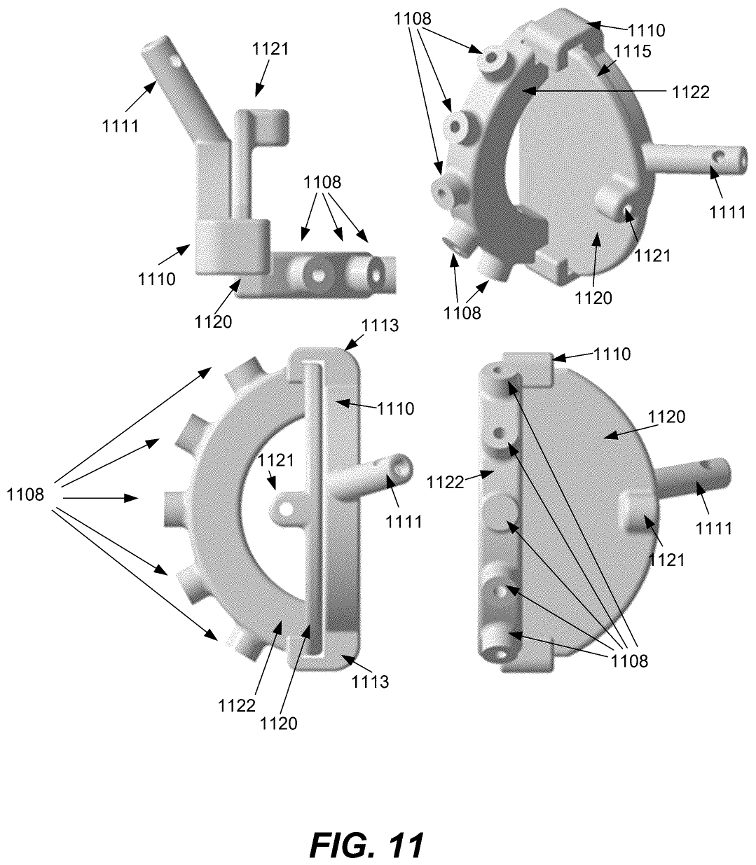

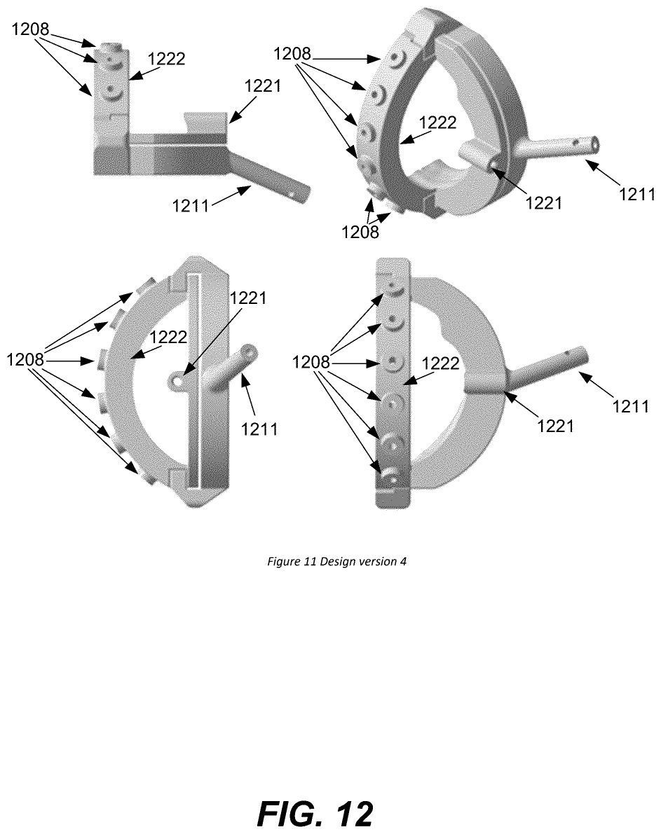

FIG. 11 shows schematic illustrations of different perspective views of another embodiment of a humeral head guide device 1100, in accordance with one or more embodiments. The humeral head guide device 1100 includes a seat 1110, a plate 1120, an armature 1122, guide wire channels 1008, one or more alignment channels 1111, and a fixation channel 1121. The humeral head guide device 1100 revises the embodiment shown in FIG. 10, improving bulkiness and the size of the cutting plate surface. An alignment channel 1111 was also improved so that the humeral head guide device 1100 could be double checked with relation to the humeral shaft and forearm. The alignment channel 1111 may be marked in a similar manner as alignment channel 1011. Relative to the humeral head guide device 1000 illustrated in FIG. 10, the humeral head guide device 1100 may include a longer alignment channel 1111 (anterior axis shaft) to help align guide for fixation and allows for insertion of a K-wire. Relative to the humeral head guide device 1000 illustrated in FIG. 10, the humeral head guide device 1100 may extend the cutting surface for better visual and instrument access superiorly and inferiorly and includes aesthetic and ergonomic improvements, including filets 1113 and chamfers 1115. Filets 113 may reduce stress concentrations, improve strength and durability, improve appearance and safety, and facilitate assembly and mating. Chamfers 1115 may reduce sharp edges and improve safety, facilitate assembly and mating, provide clearance, and improve appearance.

FIG. 12 shows schematic illustrations of different perspective views of another embodiment of a humeral head guide device 1200, in accordance with one or more embodiments. The humeral head guide device 1200 includes a seat 1210, a plate 1220, an armature 1222, guide wire channels 1208, one or more alignment channels 1211, and a fixation channel 1221. The humeral head guide device 1200 revises the embodiment shown in FIG. 11, which reduces the size of the cutting plate to reduce the profile of the humeral head guide device. The lower profile guide improves accessibility because available access is minimal during surgery. For example, relative to the embodiment shown in FIG. 11, the diameter of an alignment channel 1211 (anterior alignment shaft) is smaller. Additionally or alternatively, relative to the embodiment shown in FIG. 11, guide wire channels 1208 are made shorter fixation wire to reduce interference with soft tissues.

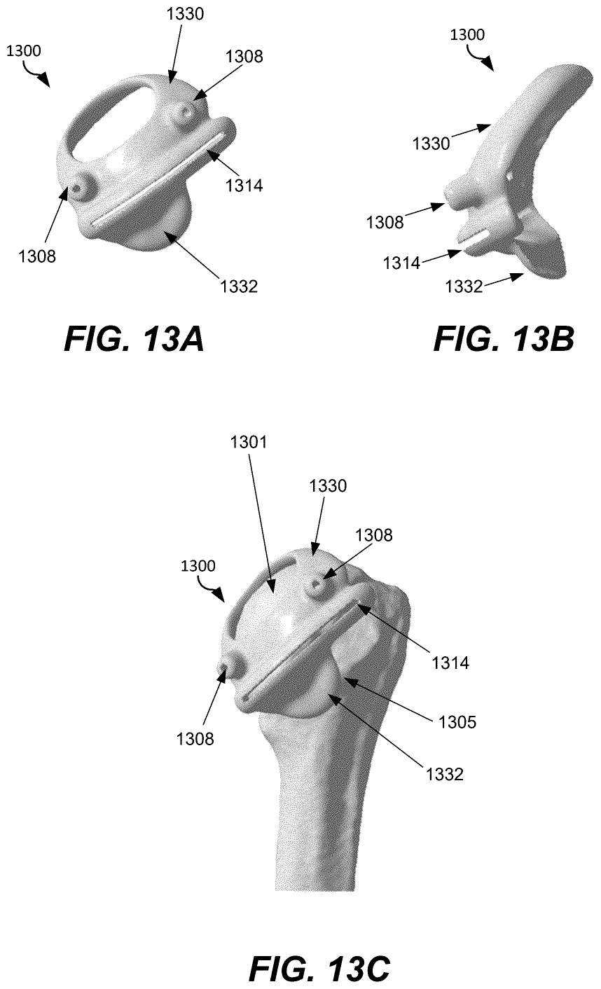

FIGS. 13A-13C show schematic illustrations of different perspective views of another embodiment of a patient-specific humeral head guide device 1300, in accordance with one or more embodiments. The humeral head guide device 1300 depicted in FIGS. 13A-13C includes a plurality of fixation wire channels 1308, a resection channel 1314, a loop style body 1330, and an extended footprint 1332. This design is based on a goal to find the anterior axis given that the scanning protocol may include only the proximal humerus (the distal humerus may be necessary to ascertain the anterior axis). The relationship between the proximal humerus anatomical landmarks (head-neck axis, bicipital groove, lesser tuberosity) and the intercondylar axis at the elbow was studied. The fixation wire channels 1308 may be configured such that fixation wires 1308 (such as K-wires) can be inserted into the humeral head 1301. In some embodiments, at least two of the fixation wire channels 1308 are configured to receive respective guide wires that are anti-parallel with respect to one another. The loop style body 1330 and the extended footprint 1332 together allows the patient-specific humeral head guide device 1300 to contour the humeral head 1301 to the lesser tuberosity 1305. For example, the loop style body 1330 has a footprint that is configured based on the contour of the humeral head 1301 (see FIG. 13C). The extended footprint 1332 is configured to inferiorly extend down to the lesser tuberosity 1305. The extended footprint 1332 may ensure at least some part of the patient-specific humeral head guide device 1300 is against the bone surface.

On the head, additional tolerance (such as approximately between 0.28-2.1 and in some examples approximately 1 mm) was given to allow for residual articulating cartilage. The design of the patient-specific humeral head guide device 1300 may avoid soft tissue attachment sites. Furthermore, the patient-specific humeral head guide device 1300 may be designed to be removed with the resected humeral head 1301 in one piece to enable faster procedures.

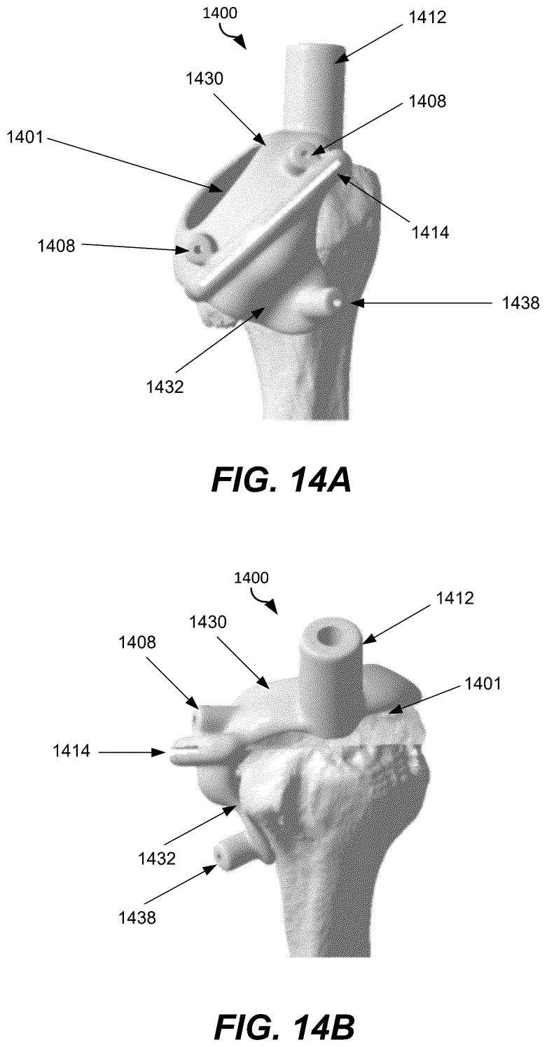

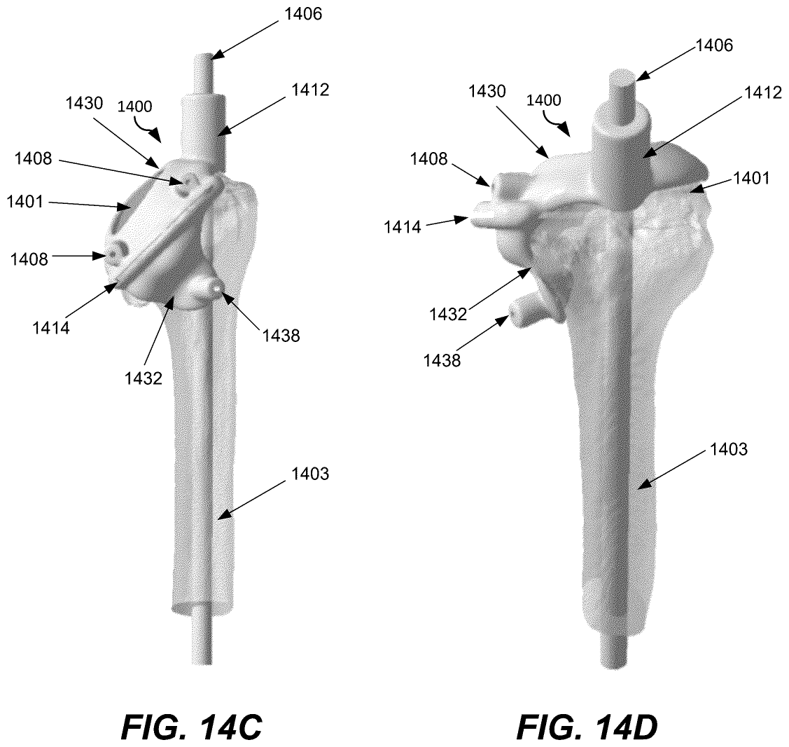

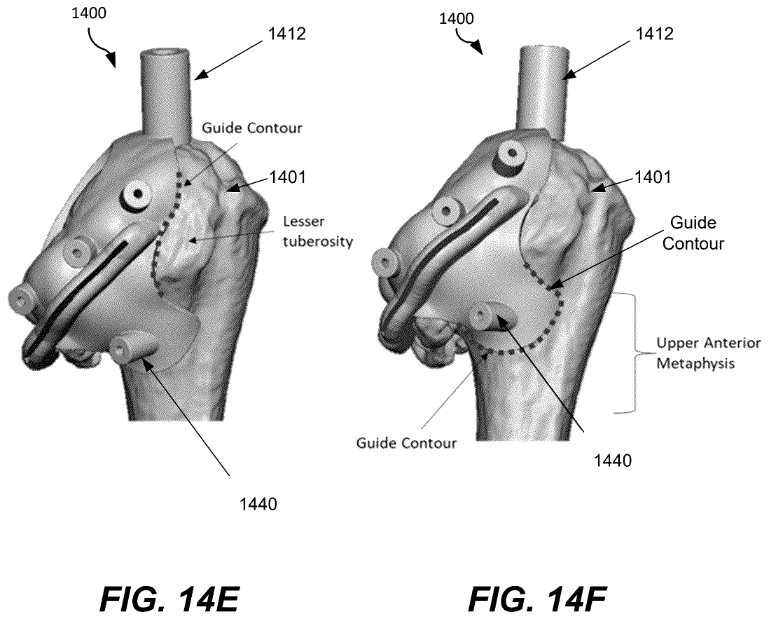

FIGS. 14A-F show schematic illustrations of different perspective views of another embodiment of a patient-specific humeral head guide device 1400, in accordance with one or more embodiments. The humeral head guide device 1400 depicted in FIGS. 14A-14F includes a plurality of fixation wire channels 1408, a reaming channel 1412, a resection channel 1414, a loop style body 1430, an extended footprint 1432, and an anterior axis fixation wire channel 1438. The anterior axis fixation wire channel 1438 is aligned to the anterior axis to allow the orientation of the humeral head guide device 1400 to be checked (such as by a surgeon) using a forearm of the patient during a procedure. The anterior axis fixation wire channel 1438 may be marked in a similar manner as alignment channel 1011 illustrated in FIG. 10. As shown in FIGS. 14C and 14D, an intermedullary rod 1406 may be inserted into the humeral head 1401 through the humerus 1403 via the reaming channel 1412, which receives the intermedullary rod 1406. As shown in FIGS. 14E and 14F, the patient-specific humeral head guide device 1400 is contoured around the shape of the humeral head 1401, as shown in dotted lines labeled (“Guide Contour”). Through contouring of the lesser tuberosity, the patient-specific humeral head guide device 1400 enables anatomical replacement when patients present intact rotator cuff. Also illustrated in FIGS. 14E and 14F is a contouring the of the upper anterior humeral metaphysis to create a fixation channel 1440 that provides a reliable fixation into the bone.

In some embodiments, additional tolerance was provided at the superolateral end of the resection channel 1414 to help avoid soft tissues. In some embodiments, the resection channel 1414 is configured so that the cutting plane is wedge-shaped to help direct the blade superiorly to minimize the amount that the humeral head guide device 1400 is extended. The humeral head guide device 1400 is configured to be removed together with the resected portion (or all) of the humeral head 1401 to speed up the procedure. In some embodiments, at least one or more of the fixation wire channels 1408 are configured to be close to the resection channel 1414 to stabilize and strengthen the cutting channel walls.

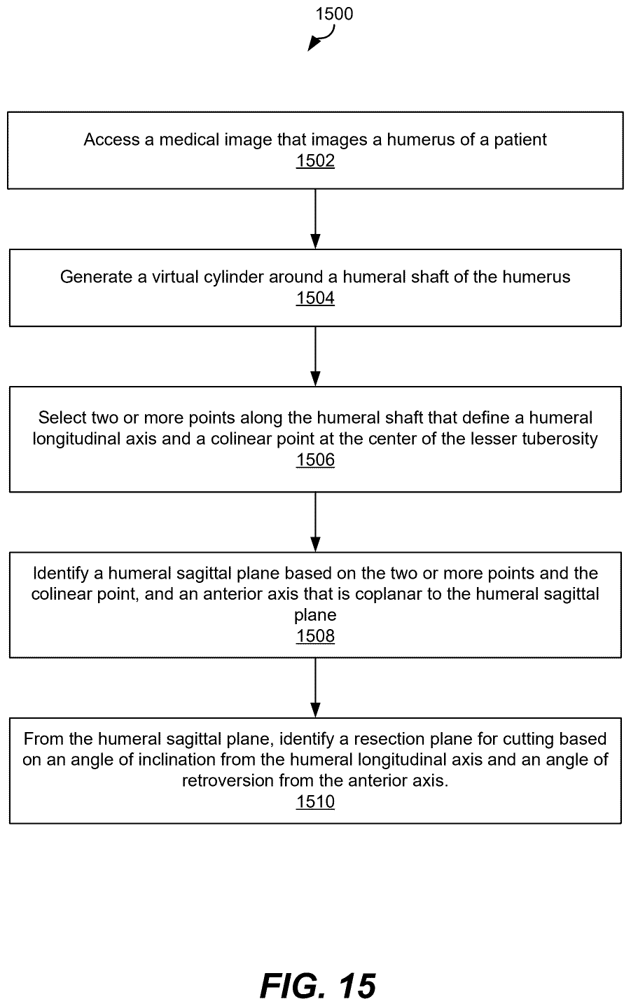

FIG. 15 illustrates a flow diagram of an example method 1500 for identifying a custom resection plane for a patient-specific humeral head guide device, in accordance with one or more embodiments. At 1502, the method 1500 may include accessing a medical image that images a humerus of a patient.

At 1504, the method 1500 may include generating a virtual cylinder around a humeral shaft of the humerus. The virtual cylinder is a conceptual shape around the humeral shaft. A system illustrated in FIGS. 1 and 2 may graphically depict the virtual cylinder via a user interface. The virtual cylinder may be a best fit shape around the humeral shaft. For example, the best fit may be generated based on one or more measured dimensions of the humeral shaft from the medical image. The dimensions may include a length, a width, and/or a height of the humeral shaft. One or more bounding boxes may be generated around the humeral object based on the one or more measurements. Each bounding box may completely surround a corresponding portion of the humeral shaft. If a single bounding box is used, then the single bounding box will completely surround the humeral shaft. The volumes (length×width×height) of the one or more bounding boxes may then be calculated along with the surface areas (a sum of the areas of all sides of each bounding box) of the one or more bounding boxes. The ratio of the volume of the one or more bounding boxes to the surface area of the one or more bounding boxes may provide an efficiency of the bounding boxes enclosing the humeral shaft. In this way, multiple sets of one or more bounding boxes may be generated to identify the best fit according to the efficiency of each set. The best fit may be the set of one or more bounding box having the highest ratio of volume to surface area.

At 1506, the method 1500 may include selecting two or more points along the humeral shaft that define a humeral longitudinal axis and a colinear point at the center of the lesser tuberosity.

At 1508, the method 1500 may include identifying a humeral sagittal plane based on the two or more points and the colinear point, and an anterior axis that is coplanar to the humeral sagittal plane.

At 1510, the method 1500 may include, from the humeral sagittal plane, identifying a resection plane for cutting based on an angle of inclination from the humeral longitudinal axis and an angle of retroversion from the anterior axis.

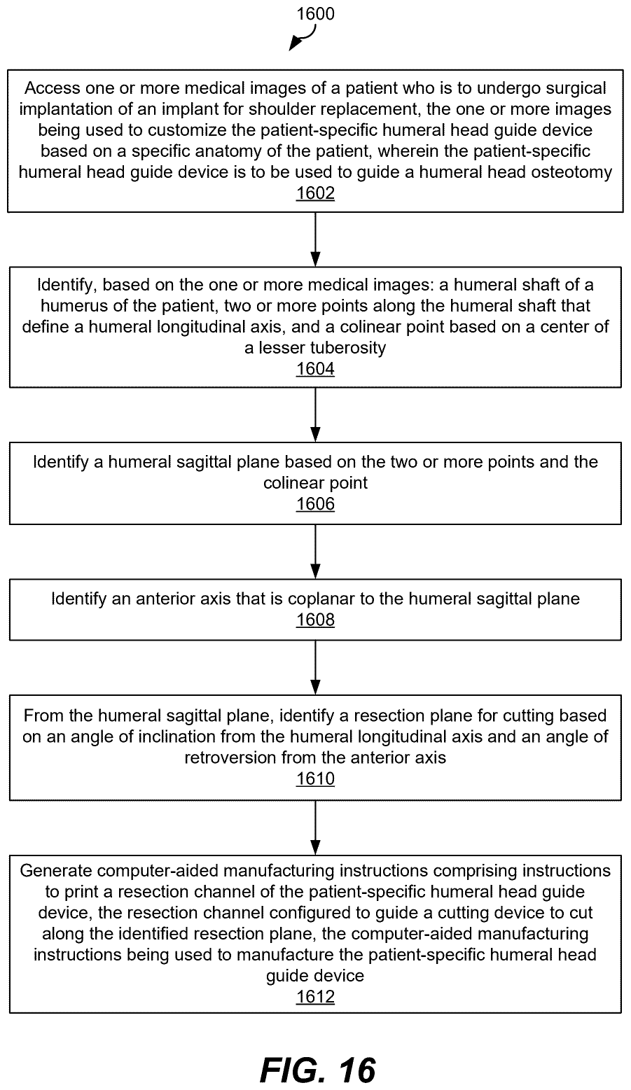

FIG. 16 illustrates a flow diagram of an example method 1600 for designing a patient-specific humeral head guide device, in accordance with one or more embodiments.

At 1602, the method 1600 may include accessing one or more medical images of a patient who is to undergo surgical implantation of an implant for shoulder replacement, the one or more images being used to customize the patient-specific humeral head guide device based on a specific anatomy of the patient, wherein the patient-specific humeral head guide device is to be used to guide a humeral head osteotomy.

At 1604, the method 1600 may include identifying, based on the one or more medical images: a humeral shaft of a humerus of the patient, two or more points along the humeral shaft that define a humeral longitudinal axis, and a colinear point based on a center of a lesser tuberosity.

At 1606, the method 1600 may include identifying a humeral sagittal plane based on the two or more points and the colinear point.

At 1608, the method 1600 may include identifying an anterior axis that is coplanar to the humeral sagittal plane.

At 1610, the method 1600 may include, from the humeral sagittal plane, identifying a resection plane for cutting based on an angle of inclination from the humeral longitudinal axis and an angle of retroversion from the anterior axis.

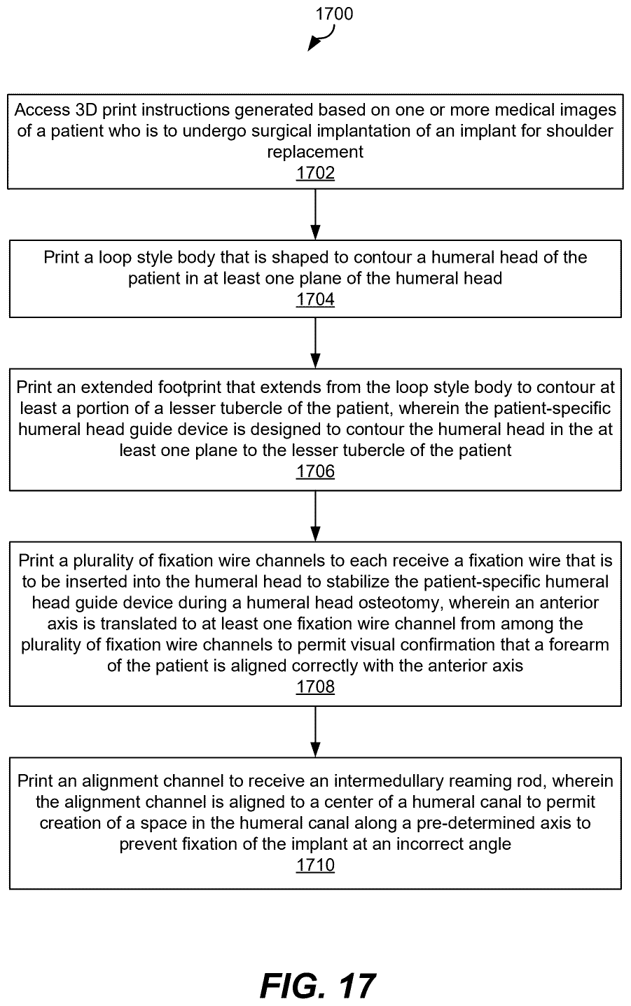

At 1612, the method 1600 may include generating computer-aided manufacturing instructions comprising instructions to print a resection channel of the patient-specific humeral head guide device. The resection channel is configured to guide a cutting device to cut along the identified resection plane. The computer-aided manufacturing instructions may be used to manufacture the patient-specific humeral head guide device. In some embodiments, when the design instructions are generated using the method 1600, the patient-specific humeral head guide device may be manufactured using the method illustrated in FIG. 17.

For example, FIG. 17 illustrates a flow diagram of an example method 1700 for manufacturing a patient-specific humeral head guide device, in accordance with one or more embodiments.

At 1702, the method 1700 may include accessing, by a 3-dimensional (3D) printer, 3D print instructions generated based on one or more medical images of a patient who is to undergo surgical implantation of an implant for shoulder replacement.

At 1704, the method 1700 may include printing, by the 3D printer based on the 3D print instructions, a loop style body that is shaped to contour a humeral head of the patient in at least one plane of the humeral head.

At 1706, the method 1700 may include printing, by the 3D printer based on the 3D print instructions, an extended footprint that extends from the loop style body to contour at least a portion of a lesser tuberosity of the patient, wherein the patient-specific humeral head guide device is designed to contour the humeral head in the at least one plane to the lesser tuberosity of the patient.

At 1708, the method 1700 may include printing, by the 3D printer based on the 3D print instructions, a plurality of fixation wire channels to each receive a fixation wire that is to be inserted into the humeral head to stabilize the patient-specific humeral head guide device during a humeral head osteotomy, wherein an anterior axis is translated to at least one fixation wire channel from among the plurality of fixation wire channels to permit visual confirmation that a forearm of the patient is aligned correctly with the anterior axis.

At 1710, the method 1700 may include printing, by the 3D printer based on the 3D print instructions, an alignment channel to receive an intermedullary reaming rod, wherein the alignment channel is aligned to a center of a humeral canal to permit creation of a space in the humeral canal along a pre-determined axis to prevent fixation of the implant at an incorrect angle.

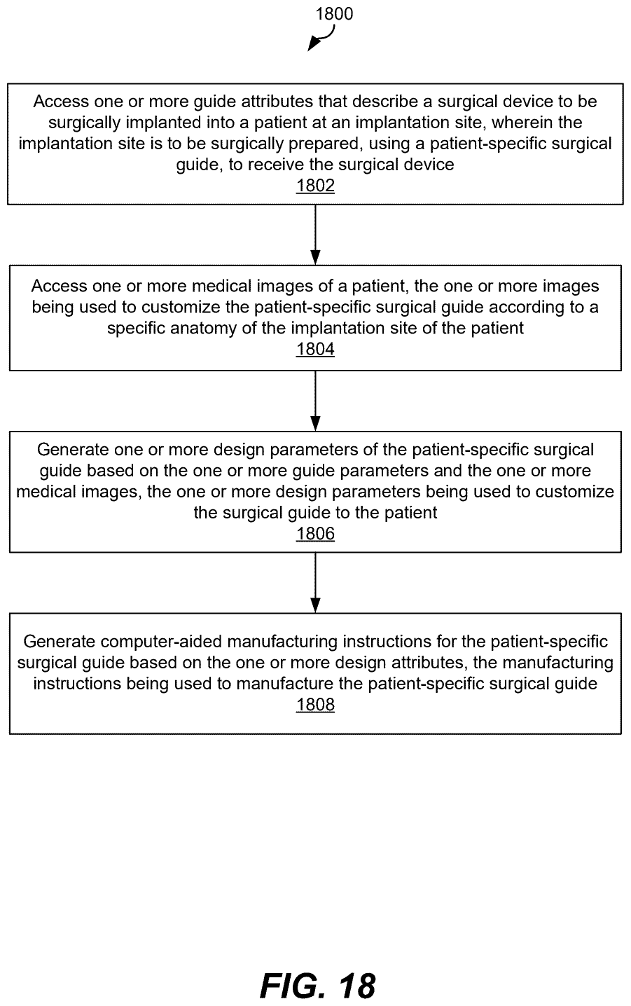

FIG. 18 illustrates a flow diagram of an example method 1800 for designing a patient-specific device, in accordance with one or more embodiments.

At 1802, the method 1800 may include accessing one or more guide attributes that describe a surgical device to be surgically implanted into a patient at an implantation site, wherein the implantation site is to be surgically prepared, using a patient-specific surgical guide, to receive the surgical device.

At 1804, the method 1800 may include accessing one or more medical images of a patient, the one or more images being used to customize the patient-specific surgical guide according to a specific anatomy of the implantation site of the patient.

At 1806, the method 1800 may include generating one or more design parameters of the patient-specific surgical guide based on the one or more guide parameters and the one or more medical images, the one or more design parameters being used to customize the surgical guide to the patient.

At 1808, the method 1800 may include generating computer-aided manufacturing instructions for the patient-specific surgical guide based on the one or more design attributes, the manufacturing instructions being used to manufacture the patient-specific surgical guide.

In some embodiments, when the design instructions are generated using the method 1800, the patient-specific humeral head guide device may be manufactured using the method illustrated in FIG. 17.

It should be noted that in some embodiments disclosed herein, the condition and geometry of the patient's anatomy may require the surgeon to be involved in the initial anterior axis alignment and cutting plane placement.

It can be understood that the systems and methods herein use medical devices as an example application to explain the concepts of the present disclosure. However, the scope of the present disclosure is not limited to medical devices. The technology platform herein can be used for controlling design and manufacturing processes associated with other user-specific devices such as mechanical devices, electrical devices, or electronic devices that have particular user-specific characteristics and may not be mass manufactured.

In some embodiments, the various computers and subsystems illustrated in FIG. 2 may include one or more computing devices that are programmed to perform the functions described herein. The computing devices may include one or more electronic storages (e.g., prediction database(s) 132, which may include training data database(s) 134, model database(s) 136, etc., or other electronic storages), one or more physical processors programmed with one or more computer program instructions, and/or other components. The computing devices may include communication lines or ports to enable the exchange of information within a network (e.g., network 150) or other computing platforms via wired or wireless techniques (e.g., Ethernet, fiber optics, coaxial cable, WiFi, Bluetooth, near field communication, or other technologies). The computing devices may include a plurality of hardware, software, and/or firmware components operating together. For example, the computing devices may be implemented by a cloud of computing platforms operating together as the computing devices.

The electronic storages may include non-transitory storage media that electronically stores information. The storage media of the electronic storages may include one or both of (i) system storage that is provided integrally (e.g., substantially non-removable) with servers or client devices or (ii) removable storage that is removably connectable to the servers or client devices via, for example, a port (e.g., a USB port, a firewire port, etc.) or a drive (e.g., a disk drive, etc.). The electronic storages may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). The electronic storage may store software algorithms, information determined by the processors, information obtained from servers, information obtained from client devices, or other information that enables the functionality as described herein.

The processors may be programmed to provide information processing capabilities in the computing devices. As such, the processors may include one or more of a digital processor, an analog processor, a digital circuit designed to process information, an analog circuit designed to process information, a state machine, and/or other mechanisms for electronically processing information. In some embodiments, the processors may include a plurality of processing units. These processing units may be physically located within the same device, or the processors may represent processing functionality of a plurality of devices operating in coordination. The processors may be programmed to execute computer program instructions to perform functions described herein of subsystems 112-120 or other subsystems. The processors may be programmed to execute computer program instructions by software; hardware; firmware; some combination of software, hardware, or firmware; and/or other mechanisms for configuring processing capabilities on the processors.

It should be appreciated that the description of the functionality provided by the different subsystems 112-120 described herein is for illustrative purposes, and is not intended to be limiting, as any of subsystems 112-120 may provide more or less functionality than is described. For example, one or more of subsystems 112-120 may be eliminated, and some or all of its functionality may be provided by other ones of subsystems 112-120. As another example, additional subsystems may be programmed to perform some or all of the functionality attributed herein to one of subsystems 112-120.

Further examples of designing and manufacturing patient-specific devices are described at Patent Cooperation Treaty (PCT) patent application no. PCT/EP2021/078024, filed on Oct. 11, 2021, which is incorporated by reference in its entirety herein for all purposes.

Although the present invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the scope of the appended claims. For example, it is to be understood that the present invention contemplates that, to the extent possible, one or more features of any embodiment can be combined with one or more features of any other embodiment.

What is claimed is:

1. A system for designing a patient-specific humeral head guide device, comprising:
a processor programmed to:
access one or more medical images of a patient who is to undergo surgical implantation of an implant for shoulder replacement, the one or more images being used to customize the patient-specific humeral head guide device based on a specific anatomy of the patient, wherein the patient-specific humeral head guide device is to be used to guide a humeral head osteotomy;
identify, based on the one or more medical images: a humeral shaft of a humerus of the patient, two or more points along the humeral shaft that define a humeral longitudinal axis, and a colinear point based on a center of a lesser tuberosity;
identify a humeral sagittal plane based on the two or more points and the colinear point;
identify an anterior axis that is coplanar to the humeral sagittal plane;
from the humeral sagittal plane, identify a resection plane for cutting based on an angle of inclination from the humeral longitudinal axis and an angle of retroversion from the anterior axis; and
generate computer-aided manufacturing instructions comprising instructions to print a resection channel of the patient-specific humeral head guide device, the resection channel configured to guide a cutting device to cut along the identified resection plane, the computer-aided manufacturing instructions being used to manufacture the patient-specific humeral head guide device.

2. The system of claim 1, wherein the computer-aided manufacturing instructions are in a 3-dimensional print code.

3. The system of claim 1, wherein the computer-aided manufacturing instructions further comprise instructions to print a loop style body that is shaped to contour a humeral head of a patient in at least one plane of the humeral head.

4. The system of claim 1, wherein the computer-aided manufacturing instructions further comprise instructions to print a loop style body that is shaped to contour a humeral head of a patient in three planes of the humeral head.

5. The system of claim 4, wherein the computer-aided manufacturing instructions further comprise instructions to print an extended footprint that extends from the loop style body to contour at least a portion of a lesser tuberosity of the patient, wherein the humeral head resection guide device is designed to contour the humeral head to the lesser tuberosity of the patient.

6. The system of claim 1, wherein the computer-aided manufacturing instructions further comprise instructions to print a plurality of fixation wire channels to each receive a fixation wire that is to be inserted into the humeral head to stabilize the humeral head resection guide device during the humeral head osteotomy.

7. The system of claim 6, wherein the anterior axis is translated to at least one fixation wire channel from among the plurality of fixation wire channels to permit visual confirmation that a forearm of the patient is aligned correctly with the anterior axis.

8. The system of claim 1, wherein the computer-aided manufacturing instructions further comprise instructions to print an alignment channel to receive an intermedullary reaming rod, wherein the alignment channel is aligned to a center of a humeral canal to permit creation of a space in the humeral canal along a pre-determined axis to prevent fixation of an implant at an incorrect angle, wherein the patient-specific humeral head guide device is used to prepare the humerus for surgical implantation of the implant for the shoulder replacement.

9. A method for designing a patient-specific humeral head guide device, comprising: accessing, by a processor, one or more medical images of a patient who is to undergo surgical implantation of an implant for shoulder replacement, the one or more images being used to customize the patient-specific humeral head guide device based on a specific anatomy of the patient, wherein the patient-specific humeral head guide device is to be used to guide a humeral head osteotomy;
identifying, by the processor, based on the one or more medical images: a humeral shaft of a humerus of the patient, two or more points along the humeral shaft that define a humeral longitudinal axis, and a colinear point based on a center of a lesser tuberosity;
identifying, by the processor, a humeral sagittal plane based on the two or more points and the colinear point;
identifying, by the processor, an anterior axis that is coplanar to the humeral sagittal plane;
from the humeral sagittal plane, identifying, by the processor, a resection plane for cutting based on an angle of inclination from the humeral longitudinal axis and an angle of retroversion from the anterior axis; and
generating, by the processor, computer-aided manufacturing instructions comprising instructions to print a resection channel of the patient-specific humeral head guide device, the resection channel configured to guide a cutting device to cut along the identified resection plane, the computer-aided manufacturing instructions being used to manufacture the patient-specific humeral head guide device.

10. A method for manufacturing a patient-specific humeral head guide device, comprising:
accessing, by a 3-dimensional (3D) printer, 3D print instructions generated based on one or more medical images of a patient who is to undergo surgical implantation of an implant for shoulder replacement;
printing, by the 3D printer based on the 3D print instructions, a loop style body that is shaped to contour a humeral head of the patient in at least one plane of the humeral head;
printing, by the 3D printer based on the 3D print instructions, an extended footprint that extends from the loop style body to contour at least a portion of a lesser tuberosity of the patient, wherein the patient-specific humeral head guide device is designed to contour the humeral head in the at least one plane to the lesser tuberosity of the patient;
printing, by the 3D printer based on the 3D print instructions, a plurality of fixation wire channels to each receive a fixation wire that is to be inserted into the humeral head to stabilize the patient-specific humeral head guide device during a humeral head osteotomy, wherein an anterior axis is translated to at least one fixation wire channel from among the plurality of fixation wire channels to permit visual confirmation that a forearm of the patient is aligned correctly with the anterior axis; and
printing, by the 3D printer based on the 3D print instructions, an alignment channel to receive an intermedullary reaming rod, wherein the alignment channel is aligned to a center of a humeral canal to permit creation of a space in the humeral canal along a pre-determined axis to prevent fixation of the implant at an incorrect angle.

11. A system for manufacturing patient-specific surgical guides, comprising:
a processor programmed to:
access one or more guide attributes that describe a surgical device to be surgically implanted into a patient at an implantation site, wherein the implantation site is to be surgically prepared, using a patient-specific surgical guide, to receive the surgical device;
access one or more medical images of a patient, the one or more images being used to customize the patient-specific surgical guide according to a specific anatomy of the implantation site of the patient;
generate one or more design parameters of the patient-specific surgical guide based on the one or more guide parameters and the one or more medical images, the one or more design parameters being used to customize the surgical guide to the patient; and
generate computer-aided manufacturing instructions for the patient-specific surgical guide based on the one or more design attributes, the manufacturing instructions being used to manufacture the patient-specific surgical guide.

12. The system of claim 11, wherein the manufacturing instructions comprises 3-dimensional (3D) print instructions that specify 3D printing of the patient-specific surgical guide.

13. The system of claim 11, wherein the implantation site comprises a humerus bone and the patient-specific surgical guide is to guide surgical removal of at least a portion of a humeral head of the humerus bone to prepare the humerus bone to receive a replacement implant.

14. A patient-specific humeral head resection guide device, comprising:
a loop style body that is shaped to contour a humeral head of a patient in at least one plane of the humeral head, wherein the patient is to receive an implant during a shoulder replacement surgery;
an extended footprint that extends from the loop style body to contour at least a portion of a lesser tuberosity of the patient, wherein the humeral head resection guide device is designed to contour the humeral head in the at least one plane to the lesser tuberosity of the patient;
a plurality of fixation wire channels (508) to each receive a fixation wire (509) that is to be inserted into the humeral head to stabilize the humeral head resection guide device during a humeral head osteotomy;
wherein an anterior axis is translated to at least one fixation wire channel (1438) from among the plurality of fixation wire channels to permit visual confirmation that a forearm of the patient is aligned correctly with the anterior axis;
an alignment channel (512) to receive an intermedullary reaming rod, wherein the alignment channel is aligned to a center of a humeral canal to permit creation of a space in the humeral canal along a pre-determined axis to prevent fixation of the implant at an incorrect angle; and
a resection channel (514) at a pre-planned cutting plane having a version and an inclination of cutting, the resection channel to receive a cutting blade during the humeral head osteotomy in which at least a portion of the humeral head is removed in preparation for an implant to be implanted in the patient.

15. The patient-specific humeral head resection guide device of claim 14, wherein the loop style body is contoured in three planes around the humeral head.

16. The humeral head resection guide device of claim 14, wherein the resection channel is configured to create a wedge shape cutting plane to direct the cutting blade superiorly.

17. A patient-specific humeral head resection guide device, comprising:
a loop style body that is shaped to contour a humeral head of a patient in at least one plane of the humeral head;
an extended footprint that extends from the loop style body to contour at least a portion of a lesser tuberosity of the patient, wherein the humeral head resection guide device is designed to contour the humeral head in the at least one plane to the lesser tuberosity of the patient;
a plurality of fixation wire channels to each receive a fixation wire that is to be inserted into the humeral head to stabilize the humeral head resection guide device during a humeral head osteotomy; and
a resection channel at a pre-planned cutting plane having a version and an inclination of cutting, the resection channel to receive a cutting blade during the humeral head osteotomy in which at least a portion of the humeral head is removed in preparation of an implant to be implanted in the patient.

18. The humeral head resection guide device of claim 17, wherein the loop style body is contoured in three planes around the humeral head.

19. The humeral head resection guide device of claim 17, wherein the resection channel is configured so that the humeral head resection guide device remains attached to the humeral head after the humeral head osteotomy so that the humeral head resection guide device is removed together with some or all of the humeral head that is removed by the humeral head osteotomy.

20. The humeral head resection guide device of claim 17, wherein at least two of the plurality of fixation wire channels are configured in an orientation to receive respective anti-parallel fixation wires.

21. The humeral head resection guide device of claim 17, wherein the resection channel is customized to a thickness of the cutting blade to be used during the humeral head osteotomy.

22. The humeral head resection guide device of claim 17, wherein the version is approximately 30 degrees and the inclination is approximately 130 degrees.

23. The humeral head resection guide device of claim 17, wherein the resection channel is configured to create a wedge shape cutting plane to direct the cutting blade superiorly.

24. The humeral head resection guide device of claim 17, wherein an anterior axis is translated to at least one fixation wire channel from among the plurality of fixation wire channels to permit visual confirmation that a forearm of the patient is aligned correctly with the anterior axis.

25. The humeral head resection guide device of claim 17, further comprising: an alignment channel to receive an intermedullary reaming rod, wherein the alignment channel is aligned to a center of a humeral canal to permit creation of a space in the humeral canal along a pre-determined axis to prevent fixation of the implant at an incorrect angle.
